(19) United States
(12) Patent Application Publication
(10) Pub. No.: US 2007/0015936 A1
(43) Pub. Date: Jan. 18, 2007

(54) Process for preparing 3-hydroxycarboxylic acids

(76) Inventors: Xiangsheng Meng, Chanhassen, MN (US); Timothy Abraham, Minnetonka, MN (US); Paris Tsobanakis, Inver Grove Heights, MN (US)

Correspondence Address: CARGILL, INCORPORATED, LAW/24, 15407 MCGINTY ROAD WEST, WAYZATA, MN, 55391, US

(21) Appl. No.: 10/546,681

(22) PCT Filed: Feb. 24, 2004
(86) PCT No.: PCT/US04/05824

§ 371 Date: Aug. 16, 2006

Related U.S. Application Data
(60) Provisional application No. 60/449,698, filed on Feb. 24, 2003.

(51) Int. Cl. C07C 51/42

(52) U.S. Cl. 562/579; 562/580

(57) ABSTRACT

Disclosed is a process for hydrating an alpha, beta-unsaturated carboxylic acid, such as acrylic acid, in water, in the presence of a catalyst selected from carbon dioxide, a sulfur oxide, a nitrogen oxide, gaseous hydrochloric acid, an inorganic or organic base having a pKa greater than 7, to prepare a 3-hydroxycarboxylic acid such as 3-hydroxypropionic acid. Also disclosed is a process for recovering 3-hydroxypropionic acid from a solution comprising the 3-hydroxypropionic acid.

### FIELD OF THE INVENTION

This invention relates to a process for preparing 3-hydroxycarboxylic acids from alpha, beta-unsaturated carboxylic acids.

### BACKGROUND OF THE INVENTION

Various methods for producing 3-hydroxycarboxylic acids are known. Included within such methods is hydrating an acrylic acid using as a catalyst perchloric acid, sulfuric acid or para-toluenesulfonic acid. Further, it is known that acrylic acid can be hydrated in the presence of a solid acid catalyst such as crystalline aluminosilicate.

### SUMMARY OF THE INVENTION

It is accordingly an object of the present invention to provide a new process for preparing 3-hydroxycarboxylic acids, particularly 3-hydroxypropionic acid.

It is a further object of this invention to provide a new process for preparing 3-hydroxycarboxylic acids in good yield.

It is a still further object of this invention to provide a new process for recovering 3-hydroxypropionic acid from a solution comprising the 3-hydroxypropionic acid.

These and other objects and advantages of the present invention will be apparent to those skilled in the art from the following detailed description and claims.

In accordance with the present invention, it has been found that the above and still further objects are achieved by hydrating an alpha, beta-unsaturated carboxylic acid, in the presence of a specific catalyst, in the presence of water, to prepare a 3-hydroxycarboxylic acid. The catalyst used in the process of the present invention is selected from a basic catalyst, that is an inorganic or organic base having a pKa value greater than 7, carbon dioxide, a sulfur oxide, a nitrogen oxide, or gaseous hydrochloric acid. Preferably, the catalyst used in the process of the present invention is selected from ammonia, carbon dioxide, a sulfur oxide such as sulfur dioxide or trioxide, a nitrogen oxide such as nitrogen dioxide, magnesium oxide, magnesium hydroxide, calcium oxide, calcium hydroxide, ammonium hydroxide, gaseous hydrochloric acid, and mixtures thereof. Most preferred for use as a catalyst is carbon dioxide.

In further accord with the present invention, there is provided a process for recovering a 3-hydroxypropionic acid from a solution comprising the 3-hydroxypropionic acid, that comprises vacuum distilling the solution using a solvent having a boiling point of at least about 140° C., under reduced pressure, at a temperature lower than 100° C., to remove more than 95% of the acrylic acid and more than 95% of water, to produce a retentate that is vacuum distilled at a temperature of from about 110° C. to about 150° C., whereby the 3-hydroxypropionic acid is recovered.

The 3-hydroxycarboxylic acids prepared by the present process are known compounds having many applications, and the products herein are useful in such applications. In particular, the 3-hydroxycarboxylic acids, such as 3-hydroxypropionic acid, are known to be useful in the preparation of polymeric materials, and as being useful intermediates in the preparation of various organic materials.

### DETAILED DESCRIPTION OF THE INVENTION

In accordance with the present invention, it has been found that a 3-hydroxycarboxylic acid is produced by hydrating an alpha, beta-unsaturated carboxylic acid in water, in the presence of a basic catalyst that is an inorganic or organic base having a pKa value greater than 7, carbon dioxide, a sulfur oxide, a nitrogen oxide or gaseous hydrochloric acid. Any sulfur oxide may be utilized in the present process, such as sulfur dioxide or sulfur trioxide. Any nitrogen oxide may be utilized herein, such as nitrogen dioxide.

Exemplary inorganic bases having a pKa value greater than 7 that are suitable for use in the present process are alkaline earth metal oxides, such as magnesium oxide or calcium oxide; alkaline earth metal hydroxides, such as magnesium hydroxide or calcium hydroxide; alkaline earth metal carbonates or bicarbonates, such as magnesium carbonate or calcium carbonate; or mixtures of the alkaline earth metal oxides, hydroxides and/or carbonates; ammonia or ammonium hydroxide; an alkali metal oxide such as sodium oxide or potassium oxide; and an alkali metal hydroxide, such as sodium hydroxide or potassium hydroxide; an alkali metal carbonate or bicarbonate such as sodium carbonate or potassium carbonate; or mixtures of any of the inorganic bases. The inorganic bases preferred for use include ammonia, magnesium oxide, magnesium hydroxide, calcium oxide, calcium hydroxide, and ammonium hydroxide.

Exemplary organic bases having a pKa value greater than 7 that are suitable for use in the present process are preferably any amine compound. The amine compounds include a primary amine having the formula RNH₂, a secondary amine having the formula R₁R₂NH, a tertiary amine R₁R₂R₃N, wherein R₁, R₂, and R₃ are individually similar or dissimilar, and represent hydrogen, a C₁-C₈ alkyl, alkynyl or alkenyl group, linear or branched, optionally substituted by halogen, alkoxy, amino, alkylamino, or hydroxyl groups, or a C₆-C₁₀ aryl group, optionally substituted by halogen, alkoxy, amino, alkylamino, or hydroxyl groups. Examples of the primary, secondary and tertiary amines are methylamine, ethylamine, butylamine, ethanolamine, heptylamine, hexylamine, tert-octylamine, dimethylamine, dibutylamine, dipentylamine, methylethanolamine, diethanolamine, methylheptylamine, methylhexylamine, trimethylamine, triethylamine, butyldiethanolamine, and tributylamine. Also suitable for use are diamino compounds having the formula H₂NRNH₂, wherein R represents a C₁-C₈ alkyl, alkynyl or alkenyl group, linear or branched, optionally substituted by halogen, alkoxy, amino, alkylamino, or hydroxyl groups, or a C₆-C₁₀ aryl group, optionally substituted by halogen, alkoxy, amino, alkylamino, or hydroxyl groups. Examples of suitable diamino compounds are methyl-1,3-propanediamine, 1,6-hexanediamine, diaminopropane, diaminobutane, diaminopentane, and diaminocyclohexane. Also suitable for use herein are triamino compounds such as triaminopyrimidine, and nitrogen heterocycles such as piperazine, pyridine, pyrrole, and triazine.

The alpha, beta-unsaturated carboxylic acid that is hydrated in the present process is a compound having the following general formula: 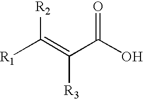
wherein R₁, R₂ and R₃ are individually similar or dissimilar, and represent hydrogen, a C₁-C₂₀ alkyl group, linear or branched, optionally substituted by halogen, alkoxy, amino, alkylamino, or hydroxyl groups, or a C₆-C₂₀ aryl group, linear or branched, optionally substituted by halogen, alkoxy, amino, alkylamino, or hydroxyl groups.

Exemplary alpha, beta-unsaturated carboxylic acids suitable for use herein are acrylic acid, methacrylic acid, 2-butenoic acid, 2-methyl-2-butenoic acid, 2-methyl-2-pentenoic acid, 3-methyl-2-butenoic acid, 2,3-dimethyl-2-butenoic acid, and cinnamic acid.

Preferably, the alpha, beta-unsaturated carboxylic acid that is hydrated in the present process is acrylic acid, resulting in the preparation of 3-hydroxypropionic acid.

Exemplary 3-hydroxycarboxylic acids that may be prepared by the present process include those having the general formula 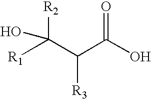
wherein R₁, R₂, and R₃ are individually similar or dissimilar, and represent hydrogen, a C₁-C₂₀ alkyl group, linear or branched, optionally substituted by halogen, alkoxy, amino, alkylamino, or hydroxyl groups, or a C₆-C₂₀ aryl group, linear or branched, optionally substituted by halogen, alkoxy, amino, alkylamino, or hydroxyl groups.

Exemplary 3-hydroxycarboxylic acids are 3-hydroxypropionic acid, 3-hydroxy-2-methylpropionic acid, 3-hydroxybutanoic acid, 3-hydroxy-2-methylbutanoic acid, 3-hydroxy-2-methylpentanoic acid, 3-hydroxy-3-methylbutanoic acid, 2,3-dimethyl-3-hydroxybutanoic acid, and 3-hydroxy-3-phenylpropionic acid.

There is no specific residence time required for the present hydration process, other than the residence time be adequate to allow the product to be produced.

The process of the present invention is carried out at a temperature ranging from about 50° C. to about 300° C., preferably from about 100° C. to about 250° C., and most preferably from about 100° C. to about 200° C.

In the embodiments of the process where the catalyst utilized in the present process is a gaseous catalyst, the process is carried out at a pressure ranging from about 0 to about 3000 psi, preferably from about 50 to about 500 psi.

In the embodiments of the process where the catalyst is a non-gaseous catalyst, the amount of catalyst utilized in the process ranges from about 1 to about 500 percent, preferably from about 5 to about 20 percent, based on the alpha, beta-unsaturated carboxylic acid that is being hydrated.

In a preferred embodiment of the present process, acrylic acid is hydrated, in water, in the presence of carbon dioxide catalyst, resulting in the preparation of 3-hydroxypropionic acid.

The hydration process of the present invention is carried out by contacting an alpha, beta-unsaturated carboxylic acid, in water, with a specified catalyst, to prepare a 3-hydroxycarboxylic acid. The process may be carried out in accordance with any manner, and the recovery of the resultant 3-hydroxycarboxylic acid is achieved according to any manner.

In more detail, when the present process is carried out utilizing acrylic acid, and a catalyst selected from carbon dioxide, a sulfur oxide, a nitrogen oxide, or gaseous hydrochloric acid, there is produced a solution comprising 3-hydroxypropionic acid. There is obtained also the ether dimer of 3-hydroxypropionic acid, identified as beta, beta′-oxydipropionic acid. In particular, the 3-hydroxypropionic acid produced herein is separated and recovered as follows. The solution comprising the 3-hydroxypropionic acid is subjected to vacuum distillation using a solvent having a boiling point of at least about 140° C., such as dodecanol. The solvent is added in an amount ranging from about 20 to about 100 weight percent, preferably about 25 weight percent, based on the solution, to produce a mixture. The mixture containing the 3-hydroxypropionic acid, and the solvent, is added into a flask connected to a vacuum line through a condenser and a dry ice trap, to remove water and acrylic acid, by distillation under reduced pressure (about 1 to 5 mm Hg) and at a temperature of below 100° C. The distillation is carried out for about one hour, after distillation of the water, to distill more than 95% of the acrylic acid from the mixture. Thereafter, the solution remaining in the flask is heated to a temperature of from about 110° C. to about 150° C., at a vacuum of about 1 to about 5 mm Hg, to distill the 3-hydroxypropionic acid along with dodecanol. The residue remaining in the container includes ether dimer of 3-hydroxypropionic acid and oligomers. The ether dimer can be recovered by any method known in the art, for example, by extraction, distillation, and the like. Moreover, any medium may be used in the extraction procedure, such as ethyl acetate.

In the instance where a basic catalyst is utilized in carrying out the hydration process of the present invention, there is produced a solution comprising a salt of a 3-hydroxypropionic acid or a mixture of the salt and the free 3-hydroxypropionic acid. To recover the 3-hydroxypropionic acid, the salt of the 3-hydroxypropionic acid is converted to the free acid, namely the 3-hydroxypropionic acid, by any method known in the art, for example, by acidification. In more detail, the 3-hydroxypropionic acid salt may be concentrated to dryness and treated with sulfuric acid. The 3-hydroxypropionic acid is then decanted away from the inorganic solids or extracted with a suitable solvent. Alternately, and preferably, the solution, now containing the 3-hydroxypropionic acid is subjected to the same procedure described hereinabove, to recover the 3-hydroxypropionic acid. The procedure for the recovery is the same as that described herein where there is produced a solution that contains 3-hydroxypropionic acid, rather than a salt of the 3-hydroxypropionic acid.

Exemplary solvents having a boiling point of at least about 140° C. that are suitable for use in the present process include alcohols, amines, amides, ethers, ketones, phosphorus esters, phosphine oxides, phosphine sulfides, alkyl sulfides, and mixtures thereof. Specific examples are dodecanol, decanol, octanol, tridodecylamine, tricaprylamine and tributyl phosphate, with dodecanol being preferred.

The invention will be more readily understood by reference to the following examples. There are, of course, many other forms of this invention which will become obvious to one skilled in the art, once the invention has been fully disclosed, and it will accordingly be recognized that these examples are given for the purpose of illustration only, and are not to be construed as limiting the scope of this invention in any way.

### EXAMPLES

In the following examples, properties of the products resulting from the process of the invention are measured using test methods as follows:

### High Pressure Liquid Chromatography (HPLC)

HPLC—The products produced by the process are analyzed using a Waters 1525 Binary HPLC pump, equipped with a Waters 717 plus Autosampler, and Waters 2410 Refractive Index and Waters 2487 Dual Lambda Absorbance detectors, having a Bio-Rad HP87-H column, 0.004 N sulfuric acid as the mobile phase, a flow rate of 0.6 ml/min, and a column temperature of 60° C.

### Gas Chromatography (GC)

GC—The protocol for the gas chromatograph was as follows: a J & W DB-WAXETR 30 m×32 mm 0.5 μm film column was used with an internal oven temperature at 90° C. with a 20° C./min increase to a final temperature of 200° C., and was maintained at 200° C. for about 12.5 minutes. The injector temperature was 200° C.

### Example 1

One hundred fifty grams of 30% acrylic acid aqueous solution was added into a 600 ml autoclave Parr reactor (available from Parr Instrument Company, Moline, Ill.). The aqueous solution in the Parr reactor was flushed with 50 psi of carbon dioxide gas three times to remove air. Then, the reactor was pressurized to 200 psi of carbon dioxide at 22° C. After that, the solution in the reactor was heated to 200° C. with stirring. The pressure of the reactor at this condition was about 400 psi. After 3 hours of mixing at 200° C., the solution in the reactor was cooled to room temperature, and then depressurized. The solution in the reactor was analyzed by HPLC and GC for the product of 3-hydroxypropionic acid, ether dimer of 3-hydroxypropionic acid, and unreacted acrylic acid. The yield of 3-hydroxypropionic acid was 54.0%, and the ether dimer was produced in a yield of 4.6%.

The 3-hydroxypropionic acid in the solution was recovered by means of vacuum distillation, utilizing dodecanol. The amount of dodecanol added was about 25 weight percent of the solution, to produce a mixture. The mixture containing the 3-hydroxypropionic acid and dodecanol was added into a flask connected to a vacuum line through a condenser and a dry ice trap, to remove water and acrylic acid by distillation under reduced pressure (about 1 to 5 mm Hg) and at a temperature of about 100° C. The distillation was carried out for about one hour, after distilling the water, to distill more than 95% of the acrylic acid from the mixture. Thereafter, the solution remaining in the flask was heated to 150° C., at a vacuum of about 1 to 5 mm Hg, to distill the 3-hydroxypropionic acid along with dodecanol.

The 3-hydroxypropionic acid obtained by the distillation contains about 5 percent acrylic dimer and about 2 percent acrylic acid. The acrylic dimer and the acrylic acid may be removed by extraction with ethyl acetate.

The residue remaining in the container includes ether dimer of 3-hydroxypropionic acid, and oligomers. The ether dimer is recovered by any method known in the art, for example, by extraction, distillation and the like.

### Example 2

The process of Example 1 was followed except that the Parr reactor was pressurized to 100 psi of carbon dioxide, rather than 200 psi. There was produced 3-hydroxypropionic acid in a yield of 53.6%, and ether dimer of 3-hydroxypropionic acid, in a yield of 4.8%.

### Example 3

The process of Example 1 was followed except that the Parr reactor was pressurized to 200 psi of carbon dioxide, the reaction temperature was 175° C., rather than 200° C., and the reaction time was 18 hours, rather than 3 hours. There was produced 3-hydroxypropionic acid in a yield of 53.6%, and ether dimer of 3-hydroxypropionic acid, in a yield of 5.8%.

### Example 4

The process of Example 1 was followed except that the Parr reactor was pressurized to 500 psi of carbon dioxide, rather than 200 psi, the reaction temperature was 170° C., rather than 200° C., and the reaction time was 18 hours, rather than 3 hours. There was produced 3-hydroxypropionic acid in a yield of 66.8%, and ether dimer of 3-hydroxypropionic acid, in a yield of 4.9%.

### Example 5

Twenty grams of 30% acrylic acid aqueous solution and 0.9 gram (15% by weight based on the acrylic acid) of magnesium oxide were charged into a 100 ml Parr reactor. The reactor was sealed and the mixture in the Parr reactor was flushed with 50 psi of nitrogen gas three times to remove air. Then, the mixture in the reactor was heated to 170° C. with stirring. After 18 hours of mixing at 170° C., the mixture in the reactor was cooled to 22° C. After separation of the solids in the mixture by centrifugation, the resultant solution was analyzed by HPLC and GC for the product of 3-hydroxypropionic acid, ether dimer of 3-hydroxypropionic acid, and unreacted acrylic acid. A yield of 3-hydroxypropionic acid of 68.7% was produced, and the ether dimer was produced in a yield of 13.1%.

### Example 6

Twenty grams of 30% acrylic acid aqueous solution and 0.9 gram (15% by weight based on the acrylic acid) of calcium oxide were charged into a 100 ml Parr reactor. The reactor was sealed and the mixture in the Parr reactor was flushed with 50 psi of nitrogen gas three times to remove air. Then, the mixture in the reactor was heated to 160° C., with stirring. After 19 hours of mixing at 160° C., the mixture in the reactor was cooled to 22° C. After separation of the solids in the mixture by centrifugation, the resultant solution was analyzed by HPLC and GC for the product of 3-hydroxypropionic acid, ether dimer of 3-hydroxypropionic acid, and unreacted acrylic acid. A yield of 3-hydroxypropionic acid of 52.0% was produced, and the ether dimer was produced in a yield of 9.0%.

### Example 7

Thirty grams of acrylic acid, 65 grams of water and 5 grams of 30% ammonium hydroxide aqueous solution were charged into a 600 ml Parr reactor. The reactor was sealed and the solution in the Parr reactor was flushed with 50 psi of nitrogen gas three times to remove air. Then, the solution in the reactor was heated to 200° C. with stirring. After 3 hours of mixing at 200° C. the solution in the reactor was cooled to 22° C. The solution in the reactor was analyzed by HPLC and GC for the product of 3-hydroxypropionic acid, ether dimer of 3-hydroxypropionic acid, and unreacted acrylic acid. A yield of 3-hydroxypropionic acid of 41.2% was produced, and the ether dimer was produced in a yield of 20.5%.

The processing conditions and the product data for Examples 1-7 are reported in the following Table 1.

TABLE 1
PREPARATION OF 3-HYDROXYPROPIONIC ACID FROM HYDRATION OF ACRYLIC ACID

| | | | | | Yield of 3- | |
|---|---|---|---|---|---|---|
| Example | Aqueous Acrylic | | Temperature | Reaction | hydroxypropionic | Yield of Ether |
| No. | Acid Solution, % | Catalyst | ° C. | Time, hours | acid, %² | Dimer, % |
| 1 | 30 | Carbon dioxide | 200 | 3 | 54.0 | 4.6 |
| | | 200 psi | | | | |
| 2 | 30 | Carbon dioxide | 200 | 3 | 53.6 | 4.8 |
| | | 100 psi | | | | |
| 3 | 30 | Carbon dioxide | 175 | 18 | 53.6 | 5.8 |
| | | 200 psi | | | | |
| 4 | 30 | Carbon dioxide | 170 | 18 | 66.8 | 4.9 |
| | | 500 psi | | | | |
| 5 | 30 | magnesium | 170 | 18 | 68.7 | 13.1 |
| | | oxide¹ 15% | | | | |
| 6 | 30 | calcium oxide¹ 15% | 160 | 19 | 52.0 | 9.0 |
| 7 | 30 | ammonium | 200 | 3 | 41.2 | 20.5 |
| | | hydroxide¹ 5% | | | | |

¹= percentage of catalyst was calculated based on weight of acrylic acid
²= yield was calculated based on 3-hydroxypropionic acid and the ester dimer of 3-hydroxypropionic acid

### Examples 8 and 9

The procedure of Example 1 is followed except that the catalyst is replaced with, in one instance, gaseous sulfur dioxide, and in another instance, gaseous sulfur trioxide, as the catalyst. It is expected that 3-hydroxypropionic acid and ether dimer of 3-hydroxypropionic acid, will be produced in both instances.

### Example 10

The procedure of Example 1 is followed except that the catalyst used is nitrogen dioxide. It is expected that 3-hydroxypropionic acid and ether dimer of 3-hydroxypropionic acid, will be produced.

### Example 11

The procedure of Example 1 is followed with the exception that gaseous hydrochloric acid is utilized as the catalyst. It is expected that 3-hydroxypropionic acid and ether dimer of 3-hydroxypropionic acid, will be produced.

### Example 12

The procedure of Example 4 is followed except that magnesium hydroxide is used as the catalyst. It is expected that 3-hydroxypropionic acid and ether dimer of 3-hydroxypropionic acid, will be produced.

### Example 13

The procedure of Example 5 is followed except that calcium hydroxide is used as the catalyst. It is expected that 3-hydroxypropionic acid and ether dimer of 3-hydroxypropionic acid will be produced.

The invention has been described above in detail with particular reference to specific embodiments thereof, but it will be understood that variations and modifications other than as specifically described herein can be effected within the spirit and scope of the invention.

We claim:

1. A process for preparing a 3-hydroxycarboxylic acid comprising hydrating an alpha, beta-unsaturated carboxylic acid of the formula 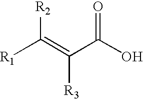
wherein R₁, R₂ and R₃ are individually similar or dissimilar, and represent hydrogen, a C₁-C₂₀ alkyl group, linear or branched, optionally substituted by halogen, alkoxy, amino, alkylamino, or hydroxyl groups, or a C₆-C₂₀ aryl group, linear or branched, optionally substituted by halogen, alkoxy, amino, alkylamino, or hydroxyl groups, in water, in the presence of a catalyst selected from the group consisting of an inorganic base having a pKa greater than 7, an organic base having a pKa greater than 7, carbon dioxide, a sulfur oxide, a nitrogen oxide, gaseous hydrochloric acid, and mixtures thereof.

2. The process according to claim 1 wherein the catalyst is selected from the group consisting of carbon dioxide, a sulfur oxide, a nitrogen oxide, gaseous hydrochloric acid, and mixtures thereof.

3. The process according to claim 1 wherein the catalyst is an inorganic base having a pKa greater than 7 or an organic base having a pKa greater than 7.

4. The process according to claim 3 wherein the catalyst is an inorganic base selected from the group consisting of ammonia, magnesium oxide, calcium oxide, magnesium hydroxide, calcium hydroxide and ammonium hydroxide.

5. The process according to claim 1 wherein the alpha, beta-unsaturated carboxylic acid is acrylic acid, and the 3-hydroxycarboxylic acid is 3-hydroxypropionic acid.

6. The process according to claim 2 wherein the catalyst is carbon dioxide.

7. The process according to claim 5 wherein the catalyst is carbon dioxide.

8. The process according to claim 1 wherein the temperature ranges from about 50° C. to about 300° C.

9. The process according to claim 1 wherein the catalyst is a gaseous catalyst and wherein the pressure ranges from about 0 to about 3000 psi.

10. The process according to claim 1 wherein the catalyst is selected from the group consisting of magnesium oxide, calcium oxide, magnesium hydroxide, calcium hydroxide, ammonium hydroxide and an amine compound, and wherein the amount of catalyst ranges from about 1 to about 500 percent, based on the weight of the alpha, beta-unsaturated carboxylic acid.

11. The process according to claim 7 wherein the temperature is about 200° C. and the pressure is about 100 psi.

12. The process according to claim 7 wherein the temperature is about 200° C. and the pressure is about 200 psi.

13. The process according to claim 7 wherein the temperature is about 170° C. and the pressure is about 500 psi.

14. The process according to claim 2 wherein the 3-hydroxycarboxylic acid is 3-hydroxypropionic acid and is prepared in solution, further comprising recovering the 3-hydroxypropionic acid by vacuum distilling the solution using a solvent having a boiling point of at least about 140° C., the solvent added in an amount of from about 20 to about 100 weight percent, based on the solution, under reduced pressure, at a temperature below 100° C. to remove more than 95% acrylic acid and more than 95% of water, thereby providing a retentate that is vacuum distilled at a temperature of from about 110° C. to about 150° C. whereby the 3-hydroxypropionic acid is recovered.

15. The process according to claim 14 wherein the solvent is dodecanol.

16. The process according to claim 3 wherein 3-hydroxycarboxylic acid is 3-hydroxypropionic acid and is prepared in a solution as a salt of the 3-hydroxypropionic acid, further comprising recovering the 3-hydroxypropionic acid by converting the salt of the 3-hydroxypropionic acid to the 3-hydroxypropionic acid and vacuum distilling the solution using a solvent having a boiling point of at least about 140° C., the solvent added in an amount of from about 20 to about 100 weight percent, based on the solution, under reduced pressure, at a temperature below 100° C. to remove more than 95% acrylic acid and more than 95% water, thereby providing a retentate that is vacuum distilled at a temperature of from about 110° C. to about 150° C., whereby the 3-hydroxypropionic acid is recovered.

17. The process according to claim 16 wherein the solvent is dodecanol.

18. A process for recovering a 3-hydroxypropionic acid from a solution comprising the 3-hydroxypropionic acid, comprising vacuum distilling the solution using a solvent having a boiling point of at least about 140° C., the solvent added in an amount of from about 20 to about 100 weight percent, based on the solution, under reduced pressure, at a temperature below 100° C. to remove more than 95% of acrylic acid and more than 95% water, thereby providing a retentate that is vacuum distilled at a temperature of from about 110° C. to about 150° C., whereby the 3-hydroxypropionic acid is recovered.

19. The process according to claim 18 wherein the solvent is dodecanol.
